(19) United States
(12) Patent Application Publication
(10) Pub. No.: US 2026/0358321 A1
(43) Pub. Date: Nov. 12, 2026

(54) FULLY AUTOMATIC HIGH-TEMPERATURE AND HIGH-PRESSURE EXPERIMENTAL APPARATUS AND METHOD FOR INTEGRATED CARBON DIOXIDE FLOODING AND SEQUESTRATION

(71) Applicants: CHINA UNIVERSITY OF PETROLEUM, Beijing (CN); UNIVERSITY OF INTERNATIONAL BUSINESS AND ECONOMICS, Beijing (CN)

(72) Inventors: Hao CHEN, Beijing (CN); Xiliang LIU, Beijing (CN); Xiucheng DONG, Beijing (CN); Yao ZHAO, Beijing (CN); Xianmin ZHOU, Beijing (CN); Haining ZHAO, Beijing (CN); Jinfeng YANG, Beijing (CN); Mingcheng NI, Beijing (CN); Peifu XU, Beijing (CN); Ziang LI, Beijing (CN); Yun SU, Beijing (CN)

(21) Appl. No.: 19/716,056

(22) Filed: Jun. 22, 2026

Related U.S. Application Data
(63) Continuation of application No. PCT/CN2026/083212, filed on Mar. 13, 2026.

(30) Foreign Application Priority Data
Mar. 21, 2025 (CN) 202510342831.5

(51) Int. Cl. G01N 33/24; G01N 15/08

(52) U.S. Cl. CPC G01N 33/24; G01N 15/088

(57) ABSTRACT

A fully automatic high-temperature and high-pressure experimental apparatus for integrated carbon dioxide (CO₂) flooding and sequestration includes a core holder system configured to hold a core sample. The core holder system is connected to a pressure measurement system and a confining pressure system. A fluid injection system is connected to the core holder system, and the fluid injection system is provided with a fluid property measurement system configured to measure fluid properties. A produced fluid separation system is connected to an outlet of the core holder system via a back pressure regulation system, and the produced fluid separation system is connected to a fluid recovery system, a fluid analysis system, and a fluid circulation system. A produced fluid measurement system is connected to the core holder system. The apparatus is applicable to evaluation of enhanced oil recovery and in-situ sequestration of CO₂, and related experimental research.

### CROSS-REFERENCE TO RELATED APPLICATION

This application is a continuation of International Patent Application No. PCT/CN2026/083212, filed on Mar. 13, 2026, which claims the priority of Chinese Patent Application No. 202510342831.5, filed on Mar. 21, 2025, both of which are herein incorporated by reference in their entirety.

### TECHNICAL FIELD

The disclosure relates to the field of petroleum extraction technologies, and more particularly to a fully automatic high-temperature and high-pressure experimental apparatus and method for integrated carbon dioxide (CO₂) flooding and sequestration.

### BACKGROUND

In the study of various technologies for enhanced oil recovery (EOR) at both laboratory and reservoir scales, CO₂ is widely used as a displacing agent in secondary and tertiary oil recovery. The EOR technology has attracted widespread attention due to its dual benefits of increasing oil and gas production and achieving in-situ geological sequestration of CO₂.

An existing Chinese patent with a patent application No. CN202411140513.2 (corresponding to a patent publication No. CN119195716A) discloses an integrated experimental apparatus and method for supercritical CO₂ fracturing, displacement, and sequestration, belonging to the technical field of natural gas development. This patent primarily utilizes supercritical CO₂ as a fracturing fluid to evaluate rock fracture pressure under triaxial in-situ stress conditions, while performing real-time monitoring and quantitative characterization of various parameters related to fracturing. The experimental apparatus is suitable for conducting fundamental experiments of supercritical CO₂ displacing methane on cubic core samples, as well as for measuring rock permeability before and after fracturing. Due to the inherent characteristics of rock fracturing experiments, the apparatus has a relatively short operational cycle and cannot be used to perform core displacement experiments such as water flooding, chemical flooding, and CO₂ flooding and sequestration. Consequently, it is unable to quantitatively characterize the injectivity of CO₂ or its oil displacement and sequestration efficiencies. Currently, this patent is intended to be applicable to the core displacement experiments using various displacement agents under high-temperature and high-pressure conditions, including chemical solution displacement experiments and water flooding experiments, and is particularly applicable to CO₂ cyclic injection experiments, such as continuous vertical and horizontal CO₂ injection, gas (e.g., CO₂)-water alternating injection, and huff-n-puff injection experiments. Moreover, because core components of this patent have excellent corrosion resistance to CO₂ and various acidic fluids, the apparatus can operate stably over long periods to meet the requirements of integrated water flooding and CO₂ displacement/sequestration experiments. In the aforementioned experiments, this patent can accurately measure various dynamic parameters, including oil, gas and water production rates, cumulative CO₂ injected and produced, inlet and outlet pressures, and the pressure difference across a test sample. At the same time, it can also quantitatively characterize key characteristic parameters, such as effective oil/water/gas permeability of the rock, displacement efficiencies of different displacement agents, and CO₂ displacement and sequestration efficiencies. Furthermore, the apparatus is suitable for quantitative evaluation of the injectivity of different displacement agents, and is particularly suitable for the CO₂ cyclic injection experiments.

Therefore, the disclosure provides a fully automatic high-temperature and high-pressure experimental apparatus and method for integrated CO₂ flooding and sequestration, so as to solve the above-described technical problems.

### SUMMARY

An objective of the disclosure is to provide a fully automatic high-temperature and high-pressure experimental apparatus and method for integrated CO₂ flooding and sequestration. It is primarily used to investigate the efficiency and influencing factors of CO₂ flooding and sequestration in conventional and unconventional oil and gas reservoirs, shale oil and gas reservoirs, and coal seams under high-temperature and high-pressure conditions. The disclosure is not only applicable to the evaluation of EOR and geological sequestration of CO₂, but can also be used to study natural depletion development, secondary oil recovery, tertiary oil recovery, and other related issues.

To achieve the above objective, the disclosure provides the following technical solutions.

The disclosure provides a fully automatic high-temperature and high-pressure experimental apparatus for integrated CO₂ flooding and sequestration, including:
a core holder system, configured to hold a core sample, where the core holder system is connected to a pressure measurement system and a confining pressure system configured to regulate confining pressure;
a fluid injection system, connected to an inlet of the core holder system and configured to inject fluid into the core holder system, where the fluid injection system is provided with a fluid property measurement system configured to measure fluid properties;
a produced fluid separation system, connected to an outlet of the core holder system via a back pressure regulation system, where the produced fluid separation system is connected to a fluid recovery system, a fluid analysis system, and a fluid circulation system; and
a produced fluid measurement system, connected to the outlet of the core holder system, and configured to measure produced fluids from the core sample in the core holder system.

In an embodiment, the core holder system includes a holder body connected to the confining pressure system. Two ends of the holder body are respectively provided with end plugs having fluid distribution. The core sample is wrapped by a sleeve provided with pressure measurement points, and is fixed inside the holder body.

In an embodiment, the confining pressure system includes a confining pressure fluid storage tank. The confining pressure fluid storage tank is connected to the holder body via a confining pressure pump. The confining pressure pump is connected to the back pressure regulation system.

In an embodiment, the fluid injection system includes a first injection pump and a second injection pump independently arranged. Each of the first injection pump and the second injection pump is connected to a first fluid tank, a second fluid tank, and a third fluid tank. The first fluid tank, the second fluid tank, and the third fluid tank are connected to the holder body and the fluid property measurement system, and the first fluid tank and the second fluid tank are connected to a pressurization system.

In an embodiment, the fluid property measurement system includes a viscometer and a densitometer arranged in parallel. The viscometer and the densitometer are configured to measure properties of a mixture of fluids discharged from the first fluid tank, the second fluid tank, and the third fluid tank.

In an embodiment, the pressurization system includes a first gas source tank and a second gas source tank independently arranged. The first gas source tank and the second gas source tank are connected to an inlet of a booster pump, and an outlet of the booster pump is connected to the first fluid tank and the second fluid tank.

In an embodiment, the produced fluid separation system includes a produced fluid separator connected to an outlet of the holder body and configured to separate gas and liquid from the produced fluids, and an outlet of the produced fluid separator is connected to the fluid recovery system, the fluid analysis system, and the fluid circulation system.

In an embodiment, the produced fluid measurement system includes a produced fluid collector and a gas volume recorder each connected to an outlet of the holder body, and the produced fluid collector and the gas volume recorder are connected to the fluid circulation system.

In an embodiment, the fluid circulation system includes a first circulation tank, a second circulation tank, and a third circulation tank arranged in parallel. The first circulation tank and the second circulation tank are connected to the first injection pump, and the third circulation tank is connected to the second injection pump.

The disclosure further provides an experimental method based on the fully automatic high-temperature and high-pressure experimental apparatus for integrated CO₂ flooding and sequestration, including:
preparing the core sample according to experimental requirements, loading the core sample into the core holder system, and connecting the confining pressure system and the pressure measurement system to the core holder system;
applying a set confining pressure to the core sample through the confining pressure system, and establishing a pore pressure of the core sample through the back pressure regulating system;
injecting brine into the core holder system through the fluid injection system, and measuring a brine permeability of the core sample under ambient conditions;
obtaining an initial water saturation and an original oil content of the core sample through a degassed oil flooding method;
restoring wettability of the core sample;
conducting a water flooding oil recovery experiment on the core sample after the restoring wettability;
conducting a CO₂ miscible flooding oil recovery and sequestration experiment on the core sample after the water flooding oil recovery experiment; and
processing collected data.

Compared with the related art, the disclosure has the following advantages and technical effects. The disclosure provides a fully automatic high-temperature and high-pressure experimental apparatus for integrated carbon dioxide CO₂ flooding and sequestration. By employing various injection modes including continuous CO₂ injection, water-alternating-gas injection, CO₂ huff and puff, chemically assisted CO₂ flooding, as well as gas-liquid or liquid-liquid two-phase flow, the apparatus is capable of studying the EOR and sequestration of CO₂. It enables real-time measurement and analysis of oil and gas compositions, evaluation of the minimum miscibility pressure between crude oil and CO₂, observation of foam phase behavior, and testing of fluid density and viscosity. Suitable for EOR and reservoir research under high-temperature and high-pressure conditions, the apparatus and method are applicable to research fields such as gas flooding, chemical flooding, and water flooding. Furthermore, they can be utilized to evaluate novel displacing agents for oil recovery, conformance control, and fracturing fluids. This achieves a multi-purpose functionality for the entire apparatus and provides valuable information unattainable by existing devices.

The disclosure achieves fully automatic control of the entire apparatus. It is applicable for the evaluation of EOR and in-situ geological sequestration of CO₂, and can also be used for experimental studies on related issues such as natural energy depletion development, secondary oil recovery, and tertiary oil recovery, thereby expanding the research field and broadening the scope of application.

### BRIEF DESCRIPTION OF DRAWINGS

Attached drawings, which constitute a part of the disclosure, are used to provide a further understanding of the disclosure. Illustrative embodiments of the disclosure and their descriptions are used to explain the disclosure and do not constitute an undue limitation on the disclosure.

FIG. 1 illustrates a structural block diagram of a fully automatic high-temperature and high-pressure experimental apparatus for integrated CO₂ flooding and sequestration according to the disclosure

FIG. 2 illustrates a schematic structural diagram of the fully automatic high-temperature and high-pressure experimental apparatus for integrated CO₂ flooding and sequestration according to the disclosure.

FIG. 3 illustrates a schematic structural diagram of a core holder system according to the disclosure.

### DESCRIPTION OF REFERENCE SIGNS

1—core sample; 2—core holder system; 3—pressure measurement system; 4—confining pressure system; 5—fluid injection system; 6—fluid property measurement system; 7—produced fluid separation system; 8—fluid recovery system; 9—fluid analysis system; 10—fluid circulation system; 11—produced fluid measurement system; 12—pressurization system; 13—imaging capture system; 14—back pressure regulation system; 15—data acquisition and control system; 201—holder body; 202—end plug; 203—pressure measurement point; 204—sleeve; 301—sensor; 401—confining pressure liquid storage tank; 402—confining pressure pump; 501—first injection pump; 502—second injection pump; 503—first fluid tank; 504—second fluid tank; 505—third fluid tank; 601—viscometer; 602—densitometer; 701—produced fluid separator; 1001—first circulation tank; 1002—second circulation tank; 1003—third circulation tank; 1101—produced fluid collector; 1102—gas volume recorder; 1201—first gas source tank; 1202—second gas source tank; 1203—booster pump; 1301—camera.

### DETAILED DESCRIPTION OF EMBODIMENTS

Technical solutions in embodiments of the disclosure will be clearly and completely described below with reference to attached drawings in the embodiments of the disclosure. It is apparent that described embodiments are only a part of the embodiments of the disclosure, rather than all of the embodiments of the disclosure. Based on the described embodiments of the disclosure, all other embodiments obtained by those skilled in the art without making creative efforts shall fall within the protection scope of the disclosure.

In order to make objectives, features, and advantages of the disclosure more apparent and understandable, the disclosure is further described in detail below with reference to the attached drawings and specific embodiments.

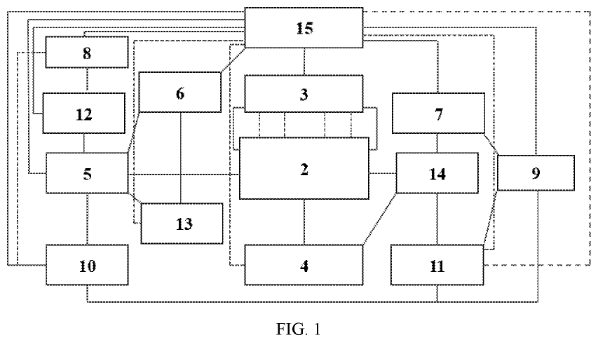

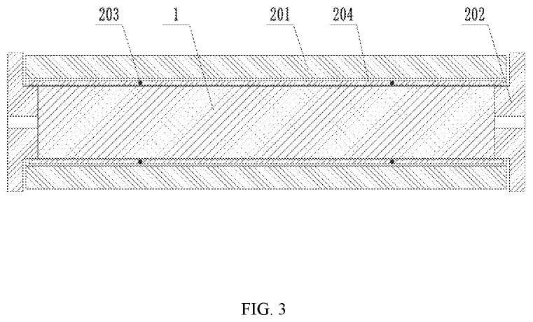

As shown in FIGS. 1-3, an embodiment of the disclosure provides a fully automatic high-temperature and high-pressure experimental apparatus for integrated CO₂ flooding and sequestration, which includes a core holder system 2, a fluid injection system 5, a produced fluid separation system 7, and a produced fluid measurement system 11.

The core holder system 2 is configured to hold a core sample 1, and the core holder system 2 is connected to a pressure measurement system 3 and a confining pressure system 4 configured to regulate confining pressure.

The fluid injection system 5 is connected to an inlet of the core holder system 2 and configured to inject fluid into the core holder system 2. The fluid injection system 5 is provided with a fluid property measurement system 6 configured to measure fluid properties.

The produced fluid separation system 7 is connected to an outlet of the core holder system 2 via a back pressure regulation system 14, and the produced fluid separation system 7 is connected to a fluid recovery system 8, a fluid analysis system 9, and a fluid circulation system 10.

The produced fluid measurement system 11 is connected to the outlet of the core holder system 2 and configured to measure produced fluids from the core sample 1 in the core holder system 2.

The disclosure provides a fully automatic high-temperature and high-pressure experimental apparatus for integrated carbon dioxide CO₂ flooding and sequestration. By employing various injection modes including continuous CO₂ injection, water-alternating-gas injection, CO₂ huff and puff, chemically assisted CO₂ flooding, as well as gas-liquid or liquid-liquid two-phase flow, the apparatus is capable of studying the EOR and sequestration of CO₂. It enables real-time measurement and analysis of oil and gas compositions, evaluation of the minimum miscibility pressure between crude oil and CO₂, observation of foam phase behavior, and testing of fluid density and viscosity. Suitable for EOR and reservoir research under high-temperature and high-pressure conditions, the apparatus and method are applicable to research fields such as gas flooding, chemical flooding, and water flooding. Furthermore, they can be utilized to evaluate novel displacing agents for oil recovery, conformance control, and fracturing fluids. This achieves a multi-purpose functionality for the entire apparatus and provides valuable information unattainable by existing devices. The disclosure achieves fully automatic control of the entire apparatus. It is applicable for the evaluation of EOR and in-situ geological sequestration of CO₂, and can also be used for experimental studies on related issues such as natural energy depletion development, secondary oil recovery, and tertiary oil recovery, thereby expanding the research field and broadening the scope of application.

Furthermore, the core holder system 2 includes a holder body 201 connected to the confining pressure system 4. Two ends of the holder body 201 are respectively provided with end plugs 202 having fluid distribution. The core sample 1 is wrapped by a sleeve 204 provided with pressure measurement points 203, and is fixed inside the holder body 201. The core holder system 2 is composed of the holder body 201, the sleeve 204 provided with the pressure measurement points 203, and the end plugs 202 each with fluid distribution lines. Each end plug 202 has distribution lines and three inlets such that fluid is uniformly injected into the core sample 1. The sleeve 204 provided with the pressure measurement points 203 is sleeved around the core sample 1 to prevent leakage of fluid within the core sample 1. After the core sample 1 is installed in place, the end plugs 202 respectively abut against the two ends of the core sample 1 to achieve fixation and allow fluid to flow into the core sample 1.

In an embodiment of the disclosure, the holder body 201 is made of a corrosion-resistant, high-temperature-resistant, and high-pressure-resistant material to ensure durability under extreme conditions.

In an embodiment of the disclosure, the sleeve 204 is designed to withstand etching solutions, surfactants, polymer solutions, and acidic gases. The pressure measurement points 203 are distributed along two sides of a central axis of the sleeve 204 and connected to the end plugs 202 at the two ends of the holder body 201 via 1/16-inch molybdenum-chromium alloy tubing. Furthermore, the tubing from the pressure measurement points 203 on the sleeve 204 is connected to the pressure measurement system 3 through the end plugs 202.

In an embodiment of the disclosure, the holder body 201 and the end plugs 202 are specifically made of a nickel-molybdenum-chromium alloy, i.e., Hastelloy C-276. This nickel-molybdenum-chromium alloy is widely recognized for its excellent corrosion resistance, mechanical strength, and durability under high-temperature and high-pressure conditions. It is capable of resisting chemical attack from supercritical CO₂ and strong acids.

In an embodiment of the disclosure, the sleeve 204 and other sealing components are specifically made of perfluoroelastomer (FFKM). FFKM is a highly specialized elastomer composed entirely of fluorinated monomers. It combines the chemical resistance of polytetrafluoroethylene (PTFE, commonly known as Teflon®) with the elastic properties of rubber, making it an ideal choice for operation under extreme chemical and thermal conditions.

In an embodiment of the disclosure, application limits of the combination of the holder body 201, the FFKM sleeve 204, and the other sealing components are determined by properties of the two materials, Hastelloy C-276 and FFKM. Based on the properties of Hastelloy C-276 and FFKM, an operating temperature of the core holder system 2 is 300 °C, and an operating pressure of the core holder system 2 is 15,000 pounds per square inch (psi), approximately 103 megapascals (MPa).

In an embodiment of the disclosure, the holder body 201 is adapted for core samples 1 of two sizes, having core diameters of 2.5 centimeters (cm) and 3.8 cm, respectively. A length of the core sample 1 with a diameter of 2.5 cm is in a range of 2.5 cm to 80 cm, and a length of the core sample 1 with a diameter of 3.8 cm is in a range of 3.8 cm to 80 cm. The length of the core sample 1 is adjustable. To accommodate the core diameters, two types of end plugs 202 for fluid distribution, with diameters of 2.5 cm and 3.8 cm respectively, are used in the holder body 201.

In an embodiment of the disclosure, the pressure measurement system 3 includes multiple sensors 301 connected to the holder body 201 to record pressure data and history related to the operation of the apparatus. Its measurement functions are as follows.

1. The pressure measurement system 3 is configured to measure inlet and outlet pressures of the core sample 1 (also referred to as upstream and downstream pressures of the core sample 1). Specifically, during a displacement experiment of the core sample 1, pressure sensors ranging from 0 to 70 MPa are used to monitor the inlet and outlet pressures at the two ends of the core sample 1. These pressure sensors are installed at inlet and outlet ends of the core sample 1. During the displacement experiment, the outlet pressure of the core sample 1 should be equal to a pore pressure, which is set by a pressure regulator.

2. The pressure measurement system 3 is configured to measure a pressure difference across the two ends of the core sample 1. Specifically, a three-stage sensor system is used to measure the pressure difference across the two ends of the core sample 1, with ranges of 0 to 0.07 MPa, 0.07 MPa to 0.7 MPa, and 0.7 Pa to 3.45 MPa. The selection of these ranges depends on the permeability of a rock (i.e., the core sample 1). When the pressure difference approaches a maximum set measurement value for a stage, the system automatically switches to a next stage. When the pressure difference approaches a maximum set measurement value of the system, the pressure difference measurement automatically stops. In this case, the pressure difference across the two ends of the core sample 1 can be measured using the pressure sensors installed at the inlet and outlet ends of the core sample 1. Each stage of the pressure testing unit is provided with a bypass valve to protect the pressure sensors. When a pressure approaches a preset pressure, the bypass valve automatically opens.

3. The pressure measurement system 3 is configured to measure pressures at the pressure measurement points 203 on the sleeve 204. Specifically, multiple pressure measurement points 203 are disposed on the sleeve 204 and connected to pressure sensors. The number of the pressure measurement points 203 depends on the design of the scheme. During the test, the pressure sensors are used to measure the pressures at different positions within the core sample 1.

4. The pressure measurement system 3 is configured to measure pressures of other pressure components. Specifically, the pressures of these pressure components include a confining pressure of the core sample 1, the pore pressure, and a back pressure.

Furthermore, the confining pressure system 4 includes a confining pressure fluid storage tank 401. The confining pressure fluid storage tank 401 is connected to the holder body 201 through a confining pressure pump 402. The confining pressure pump 402 is connected to the back pressure regulation system 14 (also referred to as back pressure regulator). The confining pressure system 4 is composed of the confining pressure pump 402 and the confining pressure fluid storage tank 401. During the displacement experiment of the core sample 1, the confining pressure is applied the confining pressure is applied by pressurizing the confining pressure fluid against the sleeve 204, ensuring that the core sample 1 is firmly fixed within the sleeve 204 to simulate the overburden pressure of the reservoir. In addition, the confining pressure pump 402 is connected to a back pressure regulation system 14 and can serve as a pressure source for the back pressure regulation system 14.

Furthermore, the fluid injection system 5 includes a first injection pump 501 and a second injection pump 502 independently arranged. Each of the first injection pump 501 and the second injection pump 502 are connected to a first fluid tank 503, a second fluid tank 504, and a third fluid tank 505. The first fluid tank 503, the second fluid tank 504, and the third fluid tank 505 are connected to the holder body 201 and the fluid property measurement system 6. The first fluid tank 503 and the second fluid tank 504 are connected to a pressurization system 12. The fluid injection system 5 includes the first injection pump 501, the second injection pump 502, and several high-temperature, high-pressure, corrosion-resistant intermediate containers. The first injection pump 501 and the second injection pump 502 inject fluid into the core sample 1 within the holder body 201 through the intermediate containers, connectors, and valves. The injection fluid system 5 can inject one or more fluids into the core sample 1 at a constant flow rate ranging from 0.0001 cubic centimeters per minute (cc/min) to 15 cc/min, with an operating pressure of up to 70 MPa.

In an embodiment of the disclosure, the intermediate containers are designed to withstand an operating pressure of up to 140 MPa.

In an embodiment of the disclosure, the number of the intermediate containers is not less than three, and they are respectively filled with CO₂, nitrogen (N₂)/brine, and crude oil.

In an embodiment of the disclosure, the intermediate containers are specifically made of a nickel-molybdenum-chromium alloy (Hastelloy C-276) for containing CO₂ and N₂, capable of resisting chemical degradation from supercritical CO₂ and strong acids. The sealing piston of each intermediate container is a metal piston made of Hastelloy C-276 and is fitted with an FFKM O-ring seal. Operating limits of each intermediate container, including the metal piston and the sealing component, are determined by a lower performance threshold between Hastelloy C-276 and FFKM.

Furthermore, the fluid property measurement system 6 includes a viscometer 601 and a densitometer 602 arranged in parallel. Fluids discharged from the first fluid tank 503, the second fluid tank 504, and the third fluid tank 505 are mixed and enter the viscometer 601 and the densitometer 602 to measure properties of mixed fluids. The fluid property measurement system 6 is composed of the high-temperature, high-pressure viscometer 601 and densitometer 602 for online measurement of viscosity and density of the injected fluid during an oil displacement process of the core sample 1 under specific reservoir conditions, and is connected to the fluid injection system 5 and the produced fluid measurement system 11. Both the viscometer 601 and the densitometer 602 can be installed either inside or outside an oven to measure the viscosity and density of the fluid under ambient conditions.

In an embodiment of the disclosure, the fluid injection system 5 of the disclosure is further connected to an imaging capture system 13. Before the fluid is injected into the core sample 1, fluid dynamic phenomena are observed and evaluated by the imaging capture system 13, such as CO₂ miscibility behavior, foam, microemulsion, phase behavior between crude oil and surfactant, and characterization of two-phase flow.

In an embodiment of the disclosure, the imaging capture system 13 includes a camera 1301 and an observation unit. The camera 1301 is equipped with a charge-coupled device (CCD) image sensor. CCD is a photosensitive integrated circuit that captures images by converting photons into electrons. The CCD image sensor decomposes image elements into pixels, and each pixel is converted into an electrical charge whose intensity is related to the intensity of the light captured by that pixel.

In an embodiment of the disclosure, the observation unit connects the imaging capture system 13 to the fluid injection system 5 and the produced fluid measurement system 11 through valves, fittings, and tubing.

Furthermore, the pressurization system 12 includes a first gas source tank 1201 and a second gas source tank 1202 independently arranged. The first gas source tank 1201 and the second gas source tank 1202 are connected to an inlet of a booster pump 1203. An outlet of the booster pump 1203 is connected to the first fluid tank 503 and the second fluid tank 504. The pressurization system 12 includes the booster pump 1203, the first gas source tank 1201, and the second gas source tank 1202, where the first gas source tank 1201 and the second gas source tank 1202 are configured to store CO₂ and N₂, respectively. The pressurization system 12 is connected to the fluid injection system 5 and the fluid circulation system 10. The pressurization system 12 is intended to increase the gas pressure to a test pressure via the booster pump 1203 and then deliver the pressurized gas to the intermediate container for storage.

In another embodiment of the disclosure, an alternative method of pressurization is to use the second injection pump 502, which can be used directly to increase the gas pressure to the test pressure. For example, if the required test pressure is 4500 psi, the pressure of a commercially available CO₂-filled cylinder is typically about 800 psi. Therefore, it is necessary to increase the gas pressure to the required test pressure.

In an embodiment of the disclosure, using the pressurization system 12 for pressurization as an example, it is assumed that the pressure of a CO₂ cylinder is 5.5 MPa and the pressure required for the experiment (i.e., the test pressure) is 31 MPa. First, CO₂ from the cylinder is transferred to a CO₂ intermediate container (the pressure in this container is 0). When the pressure in the CO₂ intermediate container reaches 800 psi (approximately 5.5 MPa), a valve on the CO₂ intermediate container is closed. Then, the second injection pump 502 is activated to deliver the CO₂ to the booster pump 1203. Subsequently, the booster pump 1203 is activated to increase the pressure to the test level, which is monitored by a pressure gauge or a pressure sensor. When the pressure reaches the pressure required for the experiment, the pressurized CO₂ is transferred to a CO₂-storing intermediate container in the fluid injection system 5. This intermediate container is already connected to the first injection pump 501 and is pre-pressurized. Once the CO₂ is transferred from the CO₂ source pressurization system 12 to the CO₂ intermediate container in the fluid injection system 5, the booster pump 1203 is activated to re-establish the test pressure.

In an embodiment of the disclosure, the first injection pump 501 can be set to a constant pressure reverse mode. A set pressure for this constant pressure mode should be 0.03 MPa lower than the experimental pressure (also referred to as test pressure). After pressure equalization, corresponding valves are closed, and the above procedure is repeated until the CO₂ intermediate container in the fluid injection system 5 is completely filled with CO₂.

In an embodiment of the disclosure, the fluid analysis system 9 includes a gas chromatograph and a high-performance liquid chromatograph, which are configured to analyze the composition and content of various gases and oils produced from the core sample 1. The gas chromatograph and the high-performance liquid chromatograph are configured to separate, identify, and quantify compounds in the produced fluids from the core sample 1. The fluid analysis system 9 is connected to the produced fluid measurement system 11 and produced fluid separation system 7 through automatic or manual valves. The analyzed liquids and gases are delivered to the fluid circulation system 10 through tubing, fittings, and valves.

Furthermore, the produced fluid separation system 7 includes a produced fluid separator 701 connected to an outlet of the holder body 201 and configured to separate gas and liquid from the produced fluids. An outlet of the produced fluid separator 701 is connected to the fluid recovery system 8, the fluid analysis system 9, and the fluid circulation system 10. The produced fluid separation system 7 includes the produced fluid separator 701 and a micro gas meter. The produced fluids discharged from the outlet end of the core sample 1 enter the produced fluid separator 701 for separation, and the micro gas meter is configured to measure the gas produced from the core sample 1 under test conditions. The produced fluid separation system 7 is connected to the outlet of the holder body 201, the fluid recovery system 8, the fluid analysis system 9, and the fluid circulation system 10 through multiple valves, fittings, and connectors.

Furthermore, the produced fluid measurement system 11 includes a produced fluid collector 1101 and a gas volume recorder 1102 connected to the outlet of the holder body 201. The produced fluid collector 1101 and the gas volume recorder 1102 are connected to the fluid circulation system 10. The produced fluid measurement system 11 is composed of the high-temperature, high-pressure produced fluid collector 1101, automatic valves, and the gas volume recorder 1102. The gas volume recorder 1102 is configured to measure gas production. The produced fluid measurement system 11 is connected to the back pressure regulation system 14, the fluid recovery system 8, and the fluid analysis system 9 through several automatic valves, tubing, and connectors. The produced fluids from the core sample 1 within the holder body 201 can be delivered to the produced fluid measurement system 11 via the back pressure regulation system 14. For example, in a liquid/gas displacement experiment using CO₂ flooding for oil recovery, the produced fluid collector 1101 can measure the produced liquid, and the gas volume recorder 1102 can measure the produced gas. The produced fluid measurement system 11 can be installed either inside or outside the oven to measure the amounts of liquid and gas produced under experimental or ambient conditions.

Furthermore, the fluid circulation system 10 includes a first circulation tank 1001, a second circulation tank 1002, and a third circulation tank 1003 arranged in parallel. The first circulation tank 1001 and the second circulation tank 1002 are connected to the first injection pump 501, and the third circulation tank 1003 is connected to the second injection pump 502. The fluid circulation system 10 includes the high-temperature and high-pressure first circulation tank 1001, second circulation tank 1002, and third circulation tank 1003. The fluid circulation system 10 is connected to the produced fluid measurement system 11, the fluid analysis system 9, and the produced fluid separation system 7 through tubing, fittings, and automatic or manual valves. Under test conditions, gases and liquids from the produced fluid measurement system 11 and the produced fluid separation system 7 enter the fluid circulation system 10. The gases and liquids are then automatically delivered by the first injection pump 501 to the intermediate containers in the fluid injection system 5 for storage and subsequent use. The gas in the third circulation tank 1003 is injected into the intermediate container by the second injection pump 502 for recycling.

In an embodiment of the disclosure, a data acquisition and control system 15 is provided. The data acquisition and control system 15 includes multiple units, such as a data acquisition and control processor, a memory, a data acquisition interface, a device control interface, and a network interface. The data acquisition and control system 15 can acquire information from various information sources of the disclosure.

The disclosure further provides an experimental method based on the fully automatic high-temperature and high-pressure experimental apparatus for CO₂ flooding and sequestration, with specific operations as follows.

The core sample 1 is prepared according to experimental requirements. Then, the core sample 1 is loaded into the core holder system 2, and the confining pressure system 4 and the pressure measurement system 3 are connected to the core holder system 2. It is assumed that dimensions, physical properties, permeability, and porosity of the core sample 1 are known. The brine-saturated core sample 1 is placed into the sleeve 204. An end plug 202 is inserted into a side of the sleeve 204 and installed into an end of the holder body 201. Then, the other end plug 202 is installed into the other end of the holder body 201. In this manner, the core sample 1, the end plugs 202, and the holder body 201 are assembled into the core holder system 2.

A set confining pressure is applied to the core sample 1 by the confining pressure system 4, and a pore pressure of the core sample 1 is established by the back pressure regulation system 14. Once the core sample 1 is placed into the holder body 201, under ambient temperature conditions, confining pressure fluid is injected by the confining pressure pump 402 into an annular space between the sleeve 204 and the holder body 201. The operation procedure is as follow. First, valves MV #45 and MV #75 are opened, the confining pressure pump 402 is activated to inject the confining pressure fluid into the annular space, and air in the annular space is vented through valve MV #75. When the confining pressure fluid begins to flow out of MV #75, indicating that the air is vented, MV #75 is closed and the set confining pressure is applied, typically 1500 psi or higher, to maintain the core sample 1 within the holder body 201 at a constant pressure. The confining pressure is measured by the sensor 301 connected to the holder body 201 through valve MV #76. All lines of the pressure measurement system 3 and inlet/outlet lines are connected to the holder body 201. Before the lines are connected, all lines are saturated with the same brine used to saturate the core sample 1. Then, the pore pressure inside the core sample 1 is established using the back pressure regulation system 14. In this embodiment, the applied pore pressure is 500 psi. During operation, brine is injected into the core sample 1 through the confining pressure pump 402 by opening a series of valves, including MV #4, six-port valve B, MV #7, MV #13, six-port valve C, MV #26, SV #9 (with SV #7 and SV #8 closed), and MV #29. The injected water then flows out of the core sample 1 to the back pressure regulation system 14. When the injection pressure exceeds the pressure of the back pressure regulation system 14, the brine flows through the back pressure regulation system 14 into the produced fluid measurement system 11. The second injection pump 502 in the fluid injection system 5 is then activated to inject brine at a constant pressure or a constant flow rate, and the required pore pressure is established by the back pressure regulation system 14. After the pore pressure stabilizes, the pressure difference across the two ends of the core sample 1 is recorded, and the brine permeability depends on the pressure difference across the two ends of the core sample 1.

Brine is injected into the core holder system 2 through the fluid injection system 5, and the brine permeability of the core sample 1 is measured under ambient conditions. After the pore pressure reaches 500 psi, the brine permeability of the core sample 1 is experimentally measured under ambient conditions. During the experimental measurement, three flow rates of 0.5, 1.0, and 2.0 cc/min are employed to inject brine into the core sample 1, and corresponding pressure differences Δp and injection flow rates are recorded to calculate the brine permeability. When measuring the brine permeability, first, brine is continuously injected into the core sample 1 at a flow rate of 0.5 cc/min. During the brine injection, once the Δp across the two ends of the core sample 1 stabilizes, the Δp and the injection flow rate are recorded. Then, the injection flow rate is increased to 1.0 cc/min, and the corresponding stabilized Δp is recorded. Finally, the injection flow rate is set to 2.0 cc/min, and the stabilized Δp across the two ends of the core sample 1 is recorded. The relationship between the pressure difference and the injection flow rate should be linear. Darcy's equation is used to calculate the brine permeability of the core sample 1.

An initial water saturation and an original oil content of the core sample 1 are established by a degassed oil flooding method. The initial water saturation and the original oil content are measured under conditions of zero pore pressure at ambient temperature and under reservoir conditions. The experimental conditions are as follows: back pressure of 500 psi, confining pressure of 1500 psi, and injection flow rates of 0.05, 0.1, 0.2, 0.4, 0.8, 1.0, and 2.0 cc/min. First, water in the core sample 1 is displaced using degassed crude oil. During the oil displacement process at each flow rate, the produced water volume and the injection pressure are recorded until no further water is produced. It should be noted that the brine in the lines from the brine injection stage should be replaced with degassed crude oil to ensure that no brine or water enters the core sample 1. The specific procedure is as follows.

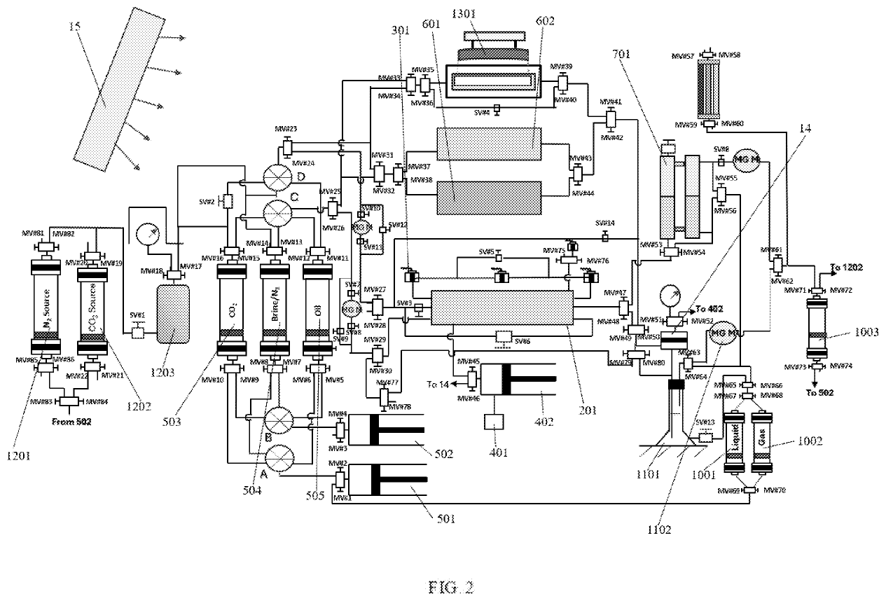

Before oil flooding, inlet and outlet lines must be cleaned with toluene or other washing solutions, and then saturated with crude oil. Assuming that the inlet and outlet lines are cleaned, the operation route for saturating the inlet lines with crude oil is as follows (as shown in FIG. 2). Starting from the second injection pump 502, the following valves are opened: MV #4, six-port valve B, MV #5, MV #11, six-port valve D, and MV #24; MV #23 is closed; SV #10 and SV #11 are closed; SV #12, MV #27, and SV #3 are opened, and MV #6 to the end plug 202 at the inlet end is closed. The crude oil passes through MV #30 and then flows out through MV #78. The operation route for saturating the outlet lines with crude oil is as follows. Starting from the second injection pump 502, the respective valves are opened or closed. Specifically, valves MV #4, six-port valve B, MV #5, MV #11, six-port valve D, MV #24, MV #27 are opened; SV #3 is closed; and valve SV #6 is opened. The crude oil then passes through the end plug 202 at the outlet end, is flowed through MV #48 and MV #49, MV #80, while MV #47, MV #50, and MV #79 are closed, and reaches the produce fluid measurement system 11, as shown in FIG. 2. After all lines, including both the inlet and outlet lines, are saturated with crude oil, the crude oil flooding experiment is ready to proceed.

According to the above-described path for saturating the lines with oil, when bypass valve SV #6 is opened, degassed crude oil is continuously injected into the system, bypassing the core sample 1. At the beginning of injection, the flow rate is set as low as possible to ensure a uniform displacement front. Once SV #3 is opened and bypass valve SV #6 is closed, oil is injected into the core sample 1, with an initial injection flow rate set to 0.05 cc/min. During the oil injection process, water in the core sample 1 is displaced over time, and the produced water volume and the pressure difference Δp are recorded. When no additional water is produced at this injection flow rate and the pressure difference is stabilized, a total volume of water displaced at the current flow rate and the stabilized Δp are recorded. The injection flow rate is then doubled to 0.1 cc/min, and the process is repeated. When no further water is produced and the Δp stabilizes at the current flow rate, the cumulative water production and pressure difference are recorded. This cycle is repeated, continuously increasing the flow rate until no further water is produced even when the flow rate is increased. In this case, a flow direction of the oil is changed to eliminate the end effect that occurs during the oil flooding process and to ensure uniform water distribution within the core sample 1. Maintaining the above-described oil injection path, valve SV #3 is closed and valve SV #6 is opened, and crude oil is injected into the core sample 1 from a right side of the holder body 201. The crude oil produced from the core sample 1 flows through valve MV #30, then sequentially through valve MV #77 and valve MV #79, and finally reaches the produced fluid measurement system 11 through the back pressure regulation system 14. During the reverse displacement process, the injection flow rate, the produced water volume, and the stabilized pressure difference are recorded, and the total produced water volume is measured. The initial water saturation and the original oil content (also referred to as original oil in place abbreviated as OOIP) are calculated using a material balance method. In addition, the effective permeability of oil under the initial water saturation is determined based on the pressure difference across the two ends of the core sample 1, the viscosity of the degassed crude oil, the dimensions of the core sample 1, and the oil injection flow rate. Darcy's equation is applied to calculate the effective permeability of oil.

The wettability of the core sample 1 is then restored. The wettability of the core sample 1 is a key factor affecting fluid dynamics, fluid distribution in pores, and oil recovery during water flooding, CO₂ flooding, and chemical flooding processes. Restoring the wettability of the core sample 1 to reservoir conditions is crucial for reservoir engineering research and for obtaining accurate experimental results.

The specific process for restoring the wettability of the core sample 1 is as follows. An oven temperature is set to a project-specific test temperature and allowed to stabilize overnight to ensure thermal equilibrium. Under an injection flow rate of 1.0 cc/min, the pore pressure and confining pressure are gradually increased in increments of 500 psi until a target pore pressure of 12,000 psi and a target confining pressure of 12,5000 psi are reached. During the pressure setting period, bypass valve SV #6 is kept open, the injection flow rate is maintained at 1.0 cc/min, and the confining pressure system 4 maintains the confining pressure at a constant value. It is assumed that the project requires live oil displacement to restore the wettability of the core sample 1 to reservoir conditions. During this process, the oil storage vessel is emptied of degassed crude oil and refilled with live crude oil. Then, with bypass valve SV #6 closed, live crude oil is used to replace the degassed crude oil. Subsequently, according to project requirements, the core sample 1 is aged in live crude oil under reservoir conditions for several weeks. During the aging process, one pore volume of live crude oil is injected daily to monitor the pressure difference and water production. At the end of the aging process, the pressure difference across the core sample 1 and the injection flow rate are recorded to calculate the effective permeability Kₑₒ at the initial water saturation. After aging with degassed crude oil and live crude oil, the core sample 1 typically exhibits mixed wettability or slight oil-wetness.

A water flooding oil recovery experiment is conducted. The water flooding oil recovery experiment is aimed at evaluating the water flooding recovery factor, residual oil saturation, water flooding characteristic parameters, and the like. This experiment is typically carried out under reservoir conditions, with a pore pressure of 12,000 psi, a confining pressure of 12,500 psi, and a temperature of 300 °C. The apparatus of the disclosure can be used to study the effects of factors such as injection flow rate, injected fluid viscosity, and water salinity on the water flooding recovery efficiency of reservoirs with different lithologies.

Before starting water flooding, the live crude oil present in the inlet, bypass, and outlet lines must be displaced using the injected water. These oil-filled lines are cleaned using brine stored in the intermediate containers, so that it is ensured that the oil produced during the water injection process comes entirely from the core sample 1. The operation procedure is as follows. First, the first injection pump 501 is started. Then, the following valves are opened: MV2, six-port valve A, MV8, MV13, six-port valve C, and MV26. SV #7 and SV #8 are closed, and SV #9 is opened. Next, MV #29 is opened, and water is directed to the inlet end plug 202 of the holder body 201. The water is then passed through SV #3 and SV #6 to the outlet end plug 202 of the holder body 201, and flows through MV #48, MV #49, and the back pressure regulation system 14. Once the water reaches the trap and no further oil is observed flowing out, it is confirmed that the oil in the lines is completely displaced.

Water injection experiment: after flushing all lines related to water flooding, the injection flow rate is adjusted to meet the engineering requirements. The operation path is as follows. Valves SV #3 and SV #6 are closed. Water is then directly injected into the core sample 1 to displace the crude oil. The water and oil produced from the core sample 1 are measured using either the produced fluid separation system 7 or the produced fluid measurement system 11. The produced fluid separation system 7 can be used under high-temperature, high-pressure conditions. The flow path for measuring oil and water production using the produced fluid separation system 7 is as follows. Crude oil from the core sample 1 first passes through MV #47, and with MV #53 opened, then enters the produced fluid separation system 7 for separation and measurement. Alternatively, for separation and measurement under ambient conditions using the trap, the flow path is as follows. Crude oil from the core sample 1 flows out through MV #48, MV #49, and MV #80, and then enters the produced fluid measurement system 11. During the water flooding experiment, the data collected include oil and water production over time, total injected water volume, and pressure difference. This information is used to evaluate the recovery factor, residual oil, and characteristic parameters of the water flooding process, such as the maximum water saturation S_{w(max)} after water flooding, the residual oil saturation after water flooding Sₒᵣ, and the water relative permeability at Sₒᵣ.

CO₂ miscible flooding oil recovery and sequestration experiments are carried out. By injecting CO₂ as a displacing agent into the waterflooded core sample 1, two benefits can be achieved: recovering some of the residual oil from the core sample 1 and achieving in-situ sequestration of CO₂ in the core sample 1 after water flooding. The purpose of injecting CO₂ into the waterflooded core sample 1 is to study the oil recovery mechanisms of CO₂ miscible flooding or immiscible flooding, and chemically assisted CO₂ injection, as well as the effects of various parameters on the production performance of CO₂ injection. The following description takes miscible flooding as an example to illustrate how to use the apparatus. In order to achieve miscible injection of crude oil and CO₂ in the formation, it is assumed that the injection pressure must be higher than the minimum miscibility pressure.

The lines are cleaned using CO₂. Specifically, before starting CO₂ miscible flooding, if water from the waterflooded core sample 1 is to be collected during CO₂ injection, the brine in the inlet, bypass, and outlet lines must be flushed out with CO₂. The procedure for cleaning the lines with CO₂ is as follows. First, a low injection flow rate is set. Then, the first injection pump 501 is started. The following valves are opened: MV #2, six-port valve A, MV #10, MV #15, six-port valve C, MV #26, SV #9, and MV #29, while SV #7, SV #8, and MV #30 are kept closed. CO₂ passes through the inlet end plug 202, SV #3, and SV #6, then through the outlet end plug 202, MV #48, MV #49, and MV #80, and reaches the produced fluid measurement system 11.

The operation procedure for CO₂ injection is as follows. The injection flow rate is set according to project requirements, and the experiment is carried out under high-temperature conditions. The CO₂ flow path is similar to that used for line cleaning. After the injection flow rate is set, the first injection pump 501 is started, and valves SV #3 and SV #6 are immediately closed. CO₂ is then injected into the core sample 1 to displace the residual oil remaining in the core sample 1 after water flooding. The produced fluids composed of oil, water, and CO₂ from the core sample 1 pass through MV #48, MV #49, and MV #80 and reaches a collector, where the oil, water, and gas are separated. The volumes of produced oil and water can be measured using a graduated tube or a high-pressure high-temperature (HPHT) imaging system. The volume of produced gas is measured using the micro gas meter or a wet gas meter. The gas flows through MV #63 to the micro gas meter for measurement. Alternatively, the produced fluid separation system 7 can be used to measure the volumes of produced oil and gas. For the gas produced, which is separated by the produced fluid separation system 7, the gas flow path is as follows. Gas from the core sample 1 flows through MV #47 and MV #53 to the produced fluid separation system 7, where the gas volume is measured. If gas composition analysis is to be performed, the gas is passed through SV #8 and MV #61 to the gas-liquid chromatography system. The valve MV #58 of this system is connected to valve MV #72 in the fluid circulation system 10, and the gas is stored in an intermediate container in the fluid circulation system 10. In addition, gas from the produced fluid separation system 7 flows through SV #8, MV #61, and MV #71 to the fluid circulation system 10.

The collected data is processed. The characteristic values associated with the above experiments include: brine permeability, initial water saturation, original oil saturation, effective permeability to oil, maximum water saturation after water flooding, residual oil saturation, effective permeability to brine at residual oil saturation, and water flooding recovery factor. For CO₂ miscible flooding, the CO₂ relative permeability at total residual oil and water saturation and the maximum CO₂ saturation are included. At the end of the CO₂ miscible injection, the CO₂ displacement efficiency and sequestration efficiency can be calculated.

Characteristic parameters based on the water flooding experiment are obtained.

Specifically, the brine permeability Kg of the core sample 1 is calculated using Darcy's law, as expressed by the following formula:

$${K}_{b} = \frac{{q}_{b} {μ}_{b} l}{Δ p A} \qquad ( 1 )$$

where K_{b} represents the brine permeability, expressed in Darcy (D); q_{b} represents the brine injection flow rate, expressed in cubic centimeter per second (cm³/s); μ_{b} represents the brine viscosity, expressed in centipoise (cP); l represents the length of the core sample 1, expressed in cm; Δp represents the pressure difference across the two ends of the core sample 1 corresponding to the brine injection flow rate, expressed in 10⁻¹ MPa; and A represents the cross-sectional area of the core sample 1, expressed in square centimeter (cm²).

During the brine injection process, the brine injection flow rate and the corresponding pressure difference of the core sample 1 are measured. With the brine viscosity, the length of the core sample 1, and the cross-sectional area of the core sample 1 remaining constant, the brine permeability can be calculated using these parameters.

The effective oil permeability Kₑₒ of the core sample 1 at the initial water saturation is calculated as per the following formula:

$${K}_{e o} = \frac{{q}_{o} {μ}_{o} l}{Δ p A} \qquad ( 2 )$$

where Kₑₒ represents the effective oil permeability at the initial water saturation, expressed in D; qₒ represents the oil injection flow rate, expressed in cm³/s; μₒ represents the oil viscosity, expressed in cP; l represents the length of the core sample 1, expressed in cm; Δp represents the pressure difference across the two ends of the core sample 1 corresponding to the oil injection flow rate, expressed in 10⁻¹ MPa; and A represents the cross-sectional area of the core sample 1, expressed in cm².

During the oil injection process, the oil injection flow rate and the corresponding pressure difference are measured. Using these measurement data, the effective oil permeability at initial water saturation can be calculated, provided that the oil viscosity, the length of the core sample 1, and the cross-sectional area of the core sample 1 remain constant.

The residual oil saturation can be obtained from the original oil content, pore volume, and produced oil volume. An expression for the residual oil saturation is as follows:

$${S}_{o r} = \frac{{V}_{o i} - {V}_{o p}}{{V}_{p}} \times 100 \qquad ( 3 )$$

An expression for the water saturation after water flooding is as follows:

S_{w(max)}=1−Sₒᵣ (4)

where Sₒᵣ represents the residual oil saturation at the end of water flooding, expressed as a percentage of pore volume (% PV) or as a percentage of OOIP (% OOIP); Vₒᵢ represents the original oil volume within the core sample 1, expressed in cm³; Vₒₚ represents the produced oil volume from the core sample 1, expressed in cm³; Vₚ represents the pore volume of the core sample 1, expressed in cm³; S_{w(max)} represents the maximum water saturation, expressed as % PV.

The effective brine permeability K_{ew} of the core sample 1 at the residual oil saturation is obtained as per the following formula:

$${K}_{e w} = \frac{{q}_{b} {μ}_{b} l}{Δ p A} \qquad ( 5 )$$

where K_{ew} represents the effective brine permeability of the core sample 1 at the residual oil saturation, expressed in D.

The water flooding oil recovery factor (also referred to as crude oil recovery factor) can be characterized by the original oil volume and the water flooding produced oil volume. The expression for the water flooding oil recovery factor is as follows:

$${R}_{fw} = \frac{{V}_{op}}{{V}_{oi}} \times 100 \% \qquad ( 6 )$$

where R_{fw} represents the water flooding oil recovery factor, expressed as % OOIP.

The main objectives of CO₂ miscible flooding are to recover the remaining oil in the core sample 1 after water flooding and to provide a site for in-situ CO₂ sequestration. Quantitative determination of the remaining oil and water contents in the water-flooded core sample CO₂ is essential for evaluating the solubility trapping mechanism of CO₂ in storage. Furthermore, after the CO₂ enhanced oil recovery stage is completed, the CO₂ injection potential can be evaluated based on various characteristic parameters of the CO₂ miscible flooding process. The characteristic parameters of CO₂ include: CO₂ flooding recovery factor; sequestration efficiency (ratio of CO₂ volume sequestered in the core sample 1 to the total volume of CO₂ injected); CO₂-oil exchange ratio (ratio of oil volume produced by CO₂ miscible flooding to the total volume of CO₂ injected); total residual oil and water saturations; effective permeability of CO₂ at total residual liquid saturation; and CO₂ injectivity coefficient.

There are two methods for calculating the CO₂ miscible flooding oil recovery factor. One method is based on the pore volume and the original oil volume of the core sample 1, and the other method is based on the remaining oil in the core sample 1 after water flooding. Using the original oil volume as the basis, the expression for calculating the recovery factor during the CO₂ injection process is as follows:

$${R}_{fCO 2} = \frac{{V}_{OCO 2}}{{V}_{oi}} \times 100 \qquad ( 7 )$$

where V_{oco2} represents the volume of oil produced during the CO₂ miscible flooding process, expressed in cm³.

Based on the remaining oil in the core sample 1 after water flooding, the expression (7) can be rewritten as follows:

$${R}_{fCO 2} = \frac{{V}_{OCO 2}}{{V}_{oi} * {S}_{or}} \times 100 \qquad ( 8 )$$

where Vₒᵢ*Sₒᵣ represents the oil volume in the core sample 1 after water flooding, expressed in cm³; and Sₒᵣ represents the residual oil saturation, expressed as % OOIP.

The CO₂ sequestration efficiency E_{sco2} is defined as a ratio of the volume of CO₂ sequestered in the core sample 1 during the CO₂ miscible flooding process to the total cumulative volume of CO₂ injected during the CO₂ miscible flooding process. The CO₂ sequestration efficiency is calculated as per the following formula:

$${E}_{s c o 2} = \frac{{V}_{C O 2}}{Σ {V}_{i n . co 2}} \times 100 \qquad ( 9 )$$

where E_{sco2} represents the CO₂ sequestration efficiency, expressed as a percentage or fraction; V_{co2} represents the volume of CO₂ retained in the core sample 1, expressed in cm³, which is the difference between the total volume of CO₂ injected and the total volume of CO₂ produced during the CO₂ miscible injection process, i.e., ΣV_{in.co2}−ΣV_{opr.co2}; ΣV_{in.co2} represents the total volume of CO₂ injected, expressed in cm³, as read by the injection pump or micro gas meter; and ΣV_{opr.co2} represents the total volume of CO₂ produced during the CO₂ miscible injection process, expressed in cm³.

The CO₂-oil exchange ratio R_{co2-oil} is defined as a ratio of the volume of oil displaced by the injected CO₂ in the core sample 1 to the total volume of CO₂ injected. An expression for the CO₂-oil exchange ratio R_{co2-oil} is as follows:

$${R}_{co 2 - oil} = \frac{{V}_{OCO 2}}{Σ {V}_{i n . co 2}} \times 100 \qquad ( 10 )$$

The total residual volume Vᵣₜ refers to a sum of volumes of residual oil and residual water in the core sample 1. An expression for the total residual volume Vᵣₜ is as follows:

Vᵣₜ=Vₒᵣ+V_{wr} (11)

where Vₒᵣ represents the volume of remaining oil in the core sample 1, expressed in cm³; and V_{wr} represents the volume of remaining water in the core sample 1, expressed in cm³. These two values can be determined by a material balance method. The remaining oil volume is a difference between the original oil volume and the total volume of oil produced from the core sample 1 during the water flooding and CO₂ flooding processes.

In a depleted reservoir after water flooding and CO₂ flooding, three phases exist: a CO₂-rich residual oil phase, a CO₂-rich water phase, and a CO₂ phase. Quantitative analysis of these three phases in the core sample 1 is essential for accurately assessing CO₂ storage capacity. Typically, in the CO₂-EOR process, only the residual oil and the maximum CO₂ saturation are evaluated. However, the residual water after CO₂ injection is often overlooked. For CO₂ storage, the CO₂-rich residual water plays an important role because a large amount of water exists in the formation, providing a medium for CO₂ dissolution. The following procedure can measure the water content after the CO₂-EOR process is completed.

At the end of water flooding, the maximum water saturation S_{w(max)} can be obtained according to the material balance method, which corresponds to the water content of the core sample 1. During the CO₂ miscible flooding process, the mixture of water, CO₂ and oil is produced simultaneously, and the production rate of each component is recorded as a function of time. Therefore, at the end of CO₂ miscible flooding, the total volume of water produced from the core sample 1 should be known and collected in the trap of the produced fluid measurement system 11. Let V_{rw-wf} be the volume of water in the core sample 1 after water flooding, which is a product of water saturation and pore volume. Let V_{pr.w-CO}_{₂} be the volume of water produced during CO₂ miscible flooding. In this case, a difference between V_{rw-wf} and V_{pr.w-CO}_{₂} represents the volume of remaining CO₂-rich water in the core sample 1 after CO₂ flooding.

Assuming that these two residual phases are not flowing in the pores of the core sample 1, the volume of CO₂ in the core sample 1 at the end of the CO₂ injection process can be calculated by the following formula:

V_{co2}=Vₚ−Vᵣₜ (12)

where V_{co2} represents the volume occupied by CO₂ in the core sample 1, expressed in cm³; Vₚ represents the pore volume of the core sample 1, expressed in cm³; and Vᵣₜ represents the total residual volume of oil and water, expressed in cm³.

Fluid injectivity refers to the ease or difficulty with which a fluid enters the formation. At a scale of the core sample 1, the injectivity index is proportional to the injection rate and inversely proportional to the pressure difference across the core sample 1. The CO₂ injectivity index I_{CO2} is expressed as:

$${I}_{C O 2} = \frac{{Q}_{CO 2}}{Δ {P}_{CO 2}} \qquad ( 13 )$$

where Q_{CO2} represents the CO₂ injection rate, expressed in cm³/min; and ΔP_{CO2} represents the pressure difference across the two ends of the core sample 1, expressed in MPa.

The relative CO₂ injection rate I_{rCO2} is also used to evaluate the degree of injection rate loss, as shown in the following formula:

$${I}_{r C O 2} = \frac{{I}_{C O 2}}{{I}_{w}} \times 100 \qquad ( 14 )$$

where I_{w} represents the water injectivity index during water injection, expressed as a percentage.

An expression for I_{w} is as follows:

$${I}_{w} = \frac{{Q}_{w}}{Δ {P}_{w}} \times 100 \qquad ( 15 )$$

where Q_{w} represents the water injection rate, expressed in cm³/min; and ΔP_{w} represents the pressure difference across the two ends of the core sample 1.

During the water flooding and CO₂ flooding processes, the parameters Q_{CO2}, Q_{w}, ΔP_{CO2} and ΔP_{w} can be obtained. Therefore, I_{CO2} and I_{rCO2} can be quantitatively calculated. Formulas (7) through (12) can be used to evaluate CO₂ sequestration potential studies.

In an embodiment of the disclosure, the fluid property measurement system 6 according to the exemplary embodiment of the disclosure includes the densitometer 602 and the viscometer 601. The fluid property measurement system 6 is capable of online measurement of the density and viscosity of a fluid before it is injected into the core sample 1, so as to ensure that the measurement results are obtained under the same test conditions as those of the core sample 1 displacement experiment. Under test conditions, both parameters can be determined in real time for any single fluid or for liquid/liquid, gas/liquid and gas/gas mixtures. The densitometer 602 and viscometer 601 can be installed in an oven together with other devices, such as the holder body 201, the imaging capture system 13 and the produced fluid separation system 7. The fluid property measurement system 6 is connected to the fluid injection system 5 and the back pressure regulating system 14 via connectors and valves. The data acquisition and control system 15 can obtain information from the fluid property measurement system 6.

Taking measurement of oil density and viscosity as an example, the fluid flow path is as follows. Specifically, the first injection pump 501 is started, valve MV #2 is opened, and valve MV #1, six-port valve A, and valve MV #6 connected to the intermediate container for oil storage are closed. The oil is passed through MV #11, six-port valve D, MV #23, MV #31 and MV #37. Then, the oil is injected into the densitometer 602 and is discharged through valve MV #43. From there, it is passed through MV #42, MV #50 and MV #80, and is introduced into the produced fluid measurement system 11 via the back pressure regulating system 14. Valves MV #37 and MV #43 are kept closed during the measurement process. For oil viscosity measurement, the operation path is followed in the same manner as that for density measurement up to MV #32. From there, the oil is introduced into the viscometer 601 through MV #32 and MV #38, and is discharged through outlet MV #44. Thereafter, the oil is passed along the same path as in the density measurement process until it is delivered to the trap.

In an embodiment of the disclosure, before CO₂ is injected into the core sample 1, the imaging capture system 13 can be used to evaluate, in real time, the dynamic behavior of injection agents such as CO₂ foaming agents, microemulsions and gels under test conditions. The imaging capture system 13 is connected to the fluid injection system 5 and the produced fluid measurement system 11 via connectors and valves. The imaging capture system 13 can be used to evaluate the minimum miscibility pressure between supercritical CO₂ and crude oil, the bubble size in CO₂ foam, and the phase behavior of surfactant solutions and crude oil in tertiary oil recovery, among others.

Typically, two injection agents are combined to form a single phase. For example, CO₂ foam is a product formed by a surfactant and CO₂ under certain conditions, and is used in research projects on chemically assisted EOR and geological sequestration of CO₂. In the imaging capture system 13, the first injection pump 501 and the second injection pump 502 can respectively inject the surfactant solution and CO₂ into the observation cell. The fluid flow path is as follows.

Before injecting the two fluids into the observation cell, the observation cell is filled with one of the two fluids, and test conditions are established. For example, if the surfactant solution and CO₂ are to be injected simultaneously, the surfactant solution may be used to fill the observation cell.

Starting from the first injection pump 501 and the second injection pump 502, the first injection pump 501 is connected through valve MV #2, six-port valve A, and valve MV #10 to an intermediate container storing CO₂. CO₂ flows out from the intermediate container, passes through MV #16, SV2, six-port valve D, MV #23, MV #34 and MV #35, and reaches the observation cell. The second injection pump 502 is used to inject the surfactant solution, with the flow path as follows: it is connected through valve MV #4, six-port valve B, and valve MV #7 to an intermediate container storing the surfactant solution (labeled as the brine/nitrogen intermediate container, as shown in FIG. 2). The surfactant solution flows out from the intermediate container, passes through MV #13, six-port valve C, MV #25, MV #33 and MV #35, and reaches the observation cell. Before the two fluids reach the observation cell, valves MV #39, MV #41 and MV #50 are opened to the back pressure regulating system 14. Under test conditions, the observation cell is filled with one of the two fluids. For example, if the surfactant solution and CO₂ are to be injected simultaneously, the surfactant solution may be used to fill the observation cell.

In an embodiment of the disclosure, the gas flow path of the pressurization system 12 is described as follows. Taking pressurization using the pressurization system 12 as an example, it is assumed that the pressure of a CO₂ cylinder is 5.5 MPa and the pressure required for the experiment is 31 MPa. First, CO₂ from the CO₂ cylinder is transferred to a CO₂ intermediate container (the pressure in this container is 0). When the pressure in the CO₂ intermediate container reaches 800 psi, the valve on the CO₂ cylinder is closed. Valve MV #3 of the second injection pump 502 is connected to valve MV #84. The second injection pump 502 is started, valves MV #3 and MV #84 are opened, and then valve MV #22 is opened, so that CO₂ flows through valve MV #19 and SV #1 to the booster pump 1203. Then, valve MV #17 is closed and valve MV #18 for pressure monitoring is opened through the booster pump 1203. The booster pump 1203 is started to increase the pressure to the test level, which is monitored by a pressure gauge or a pressure sensor. When the pressure reaches the pressure required for the experiment, valves MV #17 and MV #16 are opened, and the pressurized CO₂ is transferred to a CO₂ storage intermediate container in the fluid injection system 5. This intermediate container has been pre-pressurized and is connected to the first injection pump 501 through valve MV #2, a six-port valve, and valve MV #10. Once the CO₂ is transferred from the CO₂ source pressurization system 12 to the CO₂ intermediate container in the fluid injection system 5, SV #1 is closed and the booster pump 1203 is activated to establish the test pressure. The first injection pump 501 may be set to a constant pressure reverse mode (the set pressure should be 0.03 MPa lower than the experimental pressure). After pressure equalization, valves MV #16 and MV #17 are closed, and the above procedure is repeated until the CO₂ intermediate container in the fluid injection system 5 is fully filled.

In an embodiment of the disclosure, the heating system is composed of two ovens, which simulate test conditions by controlling temperature. The ovens are provided with front and rear doors to facilitate maintenance and replacement of components. The temperature control range is from 20 °C to 150 °C. A first oven houses the holder body 201, the densitometer 602, the viscometer 601, the produced fluid separator 701 and an observation window for fluid imaging. Multiple sensors are installed outside the first oven.

The second oven contains all of the intermediate containers, i.e., the CO₂/N₂ intermediate containers from the pressurization system 12, the CO₂, oil and brine/N₂ intermediate containers from the fluid injection system 5, and the liquid and gas recovery system intermediate containers from the fluid circulation system 10. Other components, such as the gas chromatograph and the back pressure regulating system 14, are located outside the first oven.

In an embodiment of the disclosure, the data acquisition and control system 15 includes the memory for storing data (acquisition instructions and control instructions), a control processor, the data acquisition interface, the device control interface, and control instructions. It may also include the network interface. The data acquisition and control system 15 may include a personal computer, such as a desktop computer, a laptop computer, a tablet computer, or the like.

The data acquisition and control system 15 can acquire information from various components of the disclosure, such as the fluid injection system 5, the image capture system, the pressure system, the automatic confining system, the fluid property measurement system 6, the automatic gas-liquid or liquid-liquid separator system, the micro gas metering system, the back pressure regulation system 14, the pressurization system 12, the gas-liquid chromatograph, the fluid circulation system 10, and the fluid recirculation system.

The data acquisition and control processor may include one or more processors capable of receiving and processing data from sensors of the data acquisition and control system 15. The processor may include, in some embodiments, an application-specific integrated circuit (ASIC), and the data acquisition processor may include a reduced instruction set computer (RISC) processor. In addition, the processor may include a single-core processor, a multi-core processor, and a graphics processing unit (GPU). Multiple processors may provide parallel or sequential execution of one or more techniques described in the disclosure. The processor may include, for example, a first data acquisition processor for data acquisition functions and a control processor for control functions. The processor may receive instructions and data from the memory.

The memory may include one or more tangible, non-transitory computer-readable storage media of the data acquisition and control system 15. The memory may include volatile memory, such as random access memory (RAM), and non-volatile memory, such as read-only memory (ROM), flash memory, hard disk drives, any other suitable optical, magnetic, or solid-state storage media, or combinations thereof. The memory is accessible by a processor and may store executable computer code. The executable computer code may include program instructions for implementing one or more techniques of the disclosure. For example, the executable computer code may include data acquisition instructions executable by a processor to implement one or more embodiments of the disclosure. The data acquisition instructions may include instructions for acquiring data from the sensors of the data acquisition and control system 15 via the data acquisition interface and processing the acquired data (e.g., converting the data from analog data to digital data). The processing may include comparing acquired data values (e.g., pressure values) with thresholds and providing notifications based on the comparison.

The executable computer code may include device control instructions. For example, control instructions for an oil displacement apparatus of the core sample 1 may include instructions for controlling the fluid injection system 5, the image capture system, a differential pressure system, the density and viscosity measurement system, a pressure boosting, the imaging capture system 13, the gas chromatograph, a gas-liquid or liquid-liquid separator, the back pressure regulation system 14, the automatic confining pressure system 4, or any combination thereof. Such instructions may include instructions for sending control signals, for example, sending control signals to valves of the oil displacement apparatus, displacement pumps, the back pressure regulation system 14, the automatic pneumatic control booster, and other components described in the disclosure. The processing may include comparing a data value with a threshold and providing a notification based on the comparison. The processing may include comparing a data value with a threshold and performing an action based on the comparison.

The data acquisition interface may include one or more interfaces and may provide communication between the data acquisition and control system 15 and components of the core oil displacement apparatus. For example, the data acquisition interface may include circuitry for communicating with upstream and downstream pressure sensors, differential pressure sensors, pressure sensors of injection pumps, the camera 1301 in the imaging capture system 13, the densitometer 602, the viscometer 601, the booster pump 1203, a separator, the back pressure regulator, and other components of the displacement apparatus of the core sample 1. The data acquisition interface may include a wired or wireless interface and may be used for communication via a wired network or a wireless network.

The network interface may provide communication between the data acquisition and control system 15 and other devices, such as a control system. The network interface and the data acquisition interface may be combined. The network interface may include a wired network interface card, a wireless network interface card, or a combination thereof. The network interface may include circuitry for receiving and transmitting signals to a communication network, such as an antenna system, a radio frequency transceiver, an amplifier, a tuner, an oscillator, a digital signal processor, and the like. The network interface may communicate with networks such as the Internet, an intranet, a wide area network (WAN), a local area network (LAN), a metropolitan area network (MAN), or other networks.

A display is provided. The display may include a cathode ray tube display, a liquid crystal display, an organic light emitting diode display, or other suitable displays. The display may display a user interface, and the user interface may display data acquired from the components received from the displacement apparatus of the core sample 1. The display may be a touch screen and may include or provide touch-sensitive elements through which a user may interact with the user interface. If data received from the components of the displacement apparatus of the core sample 1 satisfies a condition, the display may display a notification, such as an alarm. For example, if a pressure value obtained from a pressure sensor exceeds a threshold, a notification may be displayed.

The data acquisition and control system 15 may also provide control signals to the components of the displacement apparatus of the core sample 1. For example, it may provide the control signals to the fluid injection system 5, the pressurization system 12, the image capture system, the differential pressure system, the pressurization boosting system, the gas chromatograph, the density and viscosity measurement system, the gas-liquid or liquid-liquid separator, the back pressure regulation system 14, the gas/liquid recovery system, the gas circulation system, the automatic confining pressure system 4, or any combination thereof.

The device control interface may include one or more interfaces and may provide communication between the data acquisition and control system 15 and devices of the displacement apparatus of the core sample 1. For example, the device control interface may include circuitry for sending control signals to valves, pumps, a camera, the automatic confining pressure system 4, the back pressure regulation control system, and other devices in displacement apparatus of the core sample 1. The device control interface may include a wired or wireless interface and may be used for communication via a wired network or a wireless network.

The data acquisition and control system 15 may be coupled to input devices (e.g., one or more input devices). The input devices may include, for example, a keyboard, a mouse, a microphone, or other input devices. The input devices may enable interaction with the user interface displayed on the display. For example, an input device may provide input of values to directly or indirectly control the components of the displacement apparatus of the core sample 1.

In the description of the disclosure, it should be understood that terms “longitudinal”, “transverse”, “upper”, “lower”, “front”, “rear”, “left”, “right”, “vertical”, “horizontal”, “top”, “bottom”, “inner”, “outer”, etc., indicating orientations or positional relationships are based on orientations or positional relationships shown in the attached drawings. These terms are only for the convenience of describing the disclosure and do not indicate or imply that a referenced device or element must have a specific orientation, or be constructed and operated in a specific orientation. Therefore, they should not be construed as limitations on the disclosure.

The embodiments described above are merely descriptions of specific modes of the disclosure and are not intended to limit the scope of the disclosure. Without departing from the spirit of the disclosure, various modifications and improvements made to the technical solutions of the disclosure by those skilled in the art shall fall within the protection scope defined by the appended claims of the disclosure.

What is claimed is:

1. A fully automatic high-temperature and high-pressure experimental apparatus for integrated carbon dioxide (CO₂) flooding and sequestration, comprising:
a core holder system (2), configured to hold a core sample (1), wherein the core holder system (2) is connected to a pressure measurement system (3) and a confining pressure system (4) configured to regulate confining pressure; the core holder system (2) comprises a holder body (201) connected to the confining pressure system (4); two ends of the holder body (201) are respectively provided with end plugs (202) having fluid distribution, and the core sample (1) is wrapped by a sleeve (204) provided with pressure measurement points (203), and is fixed inside the holder body (201);
a fluid injection system, connected to an inlet of the core holder system (2) and configured to inject fluid into the core holder system (2), wherein the fluid injection system is provided with a fluid property measurement system (6) configured to measure fluid properties; the fluid injection system comprises a first injection pump (501) and a second injection pump (502) independently arranged, the first injection pump (501) is separately connected to a first fluid tank (503), a second fluid tank (504), and a third fluid tank (505), and the second injection pump (502) is separately connected to the first fluid tank (503), the second fluid tang (504), and the third fluid tank (505); the first fluid tank (503), the second fluid tank (504), and the third fluid tank (505) are connected to the holder body (201) and the fluid property measurement system (6), and the first fluid tank (503) and the second fluid tank (504) are connected to a pressurization system (12); and the fluid property measurement system (6) comprises a viscometer (601) and a densitometer (602) arranged in parallel, fluids discharged from the first fluid tank (503), the second fluid tank (504), and the third fluid tank (505) are mixed and enter the viscometer (601) and the densitometer (602) to measure properties of mixed fluids;
a produced fluid separation system (7), connected to an outlet of the core holder system (2) via a back pressure regulation system (14), wherein the produced fluid separation system (7) is connected to a fluid recovery system (8), a fluid analysis system (9), and a fluid circulation system (10), the fluid circulation system (10) comprises a first circulation tank (1001), a second circulation tank (1002), and a third circulation tank (1003) arranged in parallel, the first circulation tank (1001) and the second circulation tank (1002) are connected to the first injection pump (501), and the third circulation tank (1003) is connected to the second injection pump (502); and
a produced fluid measurement system (11), connected to the outlet of the core holder system (2), and configured to measure produced fluids from the core sample (1) in the core holder system (2).

2. The fully automatic high-temperature and high-pressure experimental apparatus for integrated CO₂ flooding and sequestration as claimed in claim 1, wherein the confining pressure system (4) comprises a confining pressure fluid storage tank (401), the confining pressure fluid storage tank (401) is connected to the holder body (201) via a confining pressure pump (402), and the confining pressure pump (402) is connected to the back pressure regulation system (14).

3. The fully automatic high-temperature and high-pressure experimental apparatus for integrated CO₂ flooding and sequestration as claimed in claim 1, wherein the pressurization system (12) comprises a first gas source tank (1201) and a second gas source tank (1202) independently arranged, the first gas source tank (1201) and the second gas source tank (1201) are connected to an inlet of a booster pump (1203), and an outlet of the booster pump (1203) is connected to the first fluid tank (503) and the second fluid tank (504).

4. The fully automatic high-temperature and high-pressure experimental apparatus for integrated CO₂ flooding and sequestration as claimed in claim 1, wherein the produced fluid separation system (7) comprises a produced fluid separator (701) connected to an outlet of the holder body (201) and configured to separate fluid and liquid from the produced fluids, and an outlet of the produced fluid separator (701) is connected to the fluid recovery system (8), the fluid analysis system (9), and the fluid circulation system (10).

5. The fully automatic high-temperature and high-pressure experimental apparatus for integrated CO₂ flooding and sequestration as claimed in claim 1, wherein the produced fluid measurement system (11) comprises a produced fluid collector (1101) and a gas volume recorder (1102) each connected to an outlet of the holder body (201), and the produced fluid collector (1101) and the gas volume recorder (1102) are connected to the fluid circulation system (10).

6. A fully automatic high-temperature and high-pressure experimental method for integrated CO₂ flooding and sequestration, implemented by the fully automatic high-temperature and high-pressure experimental apparatus for integrated CO₂ flooding and sequestration as claimed in claim 1, comprising:
preparing the core sample (1) according to experimental requirements, loading the core sample (1) into the core holder system (2), and connecting the confining pressure system (4) and the pressure measurement system (3) to the core holder system (2);
applying a set confining pressure to the core sample (1) through the confining pressure system (4), and establishing a pore pressure of the core sample (1) through the back pressure regulating system (14);
injecting brine into the core holder system (2) through the fluid injection system, and measuring a brine permeability of the core sample (1) under ambient conditions;
obtaining an initial water saturation and an original oil content of the core sample (1) through a degassed oil flooding method;
restoring wettability of the core sample (1);
conducting a water flooding oil recovery experiment on the core sample (1) after the restoring wettability;
conducting a CO₂ miscible flooding oil recovery and sequestration experiment on the core sample (1) after the water flooding oil recovery experiment; and
processing collected data.
